(19) United States
(12) Patent Application Publication
(10) Pub. No.: US 2026/0137428 A1
(43) Pub. Date: May 21, 2026

(54) COUPLING DEVICE FOR COUPLING A ROD TO A BONE ANCHORING ELEMENT

(71) Applicants: Timo BIEDERMANN, Trossingen (DE); Achim SCHÜNEMANN, Villingen-Schwenningen (DE); Bernd FISCHER, Friedenweiler (DE)

(72) Inventors: Timo BIEDERMANN, Trossingen (DE); Achim SCHÜNEMANN, Villingen-Schwenningen (DE); Bernd FISCHER, Friedenweiler (DE)

(21) Appl. No.: 19/391,362

(22) Filed: Nov. 17, 2025

Related U.S. Application Data
(60) Provisional application No. 63/721,728, filed on Nov. 18, 2024.

(30) Foreign Application Priority Data
Nov. 18, 2024 (EP) 24 213 714.9

(51) Int. Cl. A61B 17/70; A61B 17/00

(52) U.S. Cl. CPC A61B 17/7032; A61B 17/7035; A61B 2017/00477

(57) ABSTRACT

A coupling device for coupling a rod to a bone anchoring element includes a receiving part having a rod receiving portion with a recess that extends downwardly to a bottom for receiving the rod, and a flexible head receiving portion defining a seat to pivotably hold and clamp a head of the bone anchoring element therein, and a locking ring positionable around the head receiving portion and configured to exert a force on the head receiving portion to lock the head. A passage in the receiving part includes a widening section that widens while extending upwardly from a lower end closer to the seat to an upper end closer but positioned axially lower than the bottom of the recess, where an inner diameter of the upper end of the widening section is greater than an inner diameter of another section of the passage located above the bottom of the recess.

### CROSS-REFERENCE TO RELATED APPLICATION(S)

The present application claims priority to and the benefit of U.S. Provisional Ser. No. 63/721,728, filed Nov. 18, 2024, the contents of which are hereby incorporated by reference in their entirety, and claims priority from European Patent Application EP 24 213 714.9, filed Nov. 18, 2024, the contents of which are hereby incorporated by reference in their entirety.

### BACKGROUND

### Field

The present application relates to a coupling device for coupling a rod to a bone anchoring element. In particular, the present application relates to a coupling device that forms part of a bone anchoring device, as well as a bone anchoring device including such a coupling device and bone anchoring element.

### Description of Related Art

In spinal surgery, often multiple segments of the spinal column have to be corrected and/or stabilized using a spinal rod and polyaxial bone anchors. During such a procedure, repeated adjustments of the bone anchoring elements and the rod relative to receiving parts of respective polyaxial bone anchoring devices may become necessary.

Usually, a polyaxial bone anchoring device includes a coupling device and a bone anchoring element with a head that is pivotably received in the coupling device and that can be locked at a desired angle relative to the coupling device. The coupling device also receives a rod that is configured to connect the polyaxial bone anchoring device to other bone anchoring devices.

U.S. Pat. No. 10,258,383 B2 describes a polyaxial bone anchoring device including a receiving part for coupling a rod to a bone anchoring element, the receiving part including a rod receiving portion, a head receiving portion for accommodating a head of the bone anchoring element, and a locking ring configured to be arranged around the head receiving portion. The locking ring can assume a locking position where the locking ring exerts a force on the head receiving portion of the receiving part to lock an inserted head, and can be moved relative to the receiving part from the locking position towards a position where the inserted head is pivotable. With such a polyaxial bone anchoring device and instrument, a temporary locking and unlocking of the head in the receiving part can be carried out several times without using a separate locking element.

U.S. Pat. No. 11,213,323 B1 describes a coupling device including a receiving part having a head receiving portion for pivotably receiving a head of a bone anchor and a rod receiving portion defining a recess for receiving a rod, the rod receiving portion having a first engagement surface for engaging an instrument. The coupling device further includes a locking ring positionable around the head receiving portion and having a second engagement surface for engaging the instrument. With the instrument, the locking ring can be moved into a pre-locking position after placement of the coupling device onto the head of the bone anchor. This prevents removal of the coupling device from the head once the coupling device has been connected to the head. The coupling device and the instrument can be used for in-situ placement of the coupling device on a bone anchor that is already inserted into bone.

### SUMMARY

In particular during in-situ placement of a coupling device and during manipulating steps carried out during surgery, it is important for a head of a bone anchoring element to be safely clamped by the coupling device, and where at the same time a force necessary for mounting the coupling device on the head is moderate.

It is an object of the invention to provide a coupling device for coupling a bone anchoring element to a rod, in particular in a polyaxial manner, and a polyaxial bone anchoring device including such a coupling device which has increased strength and stability.

A coupling device for coupling a rod to a bone anchoring element that has a shank and a head according to an embodiment of the invention includes a receiving part having a first end, a second end, a central axis extending between the first end and the second end, and a passage extending from the first end to the second end. The receiving part further includes a rod receiving portion having a recess defining a channel for receiving the rod, the recess extending from the first end towards the second end to a bottom and forming two free legs, and a head receiving portion open to the second end and including a seat portion for pivotably receiving the head of the bone anchoring element, the head receiving portion being flexible for inserting and clamping the head. The coupling device further includes a locking ring configured to be arranged around the head receiving portion. When the locking ring is around the head receiving portion, the locking ring can assume a locking position where the locking ring exerts a force on the head receiving portion to lock an inserted head. The passage has a widening section that widens from the seat portion towards the bottom of the recess.

Due to the widening section, the coupling device may have an increased strength or stiffness without requiring an increased force necessary for mounting the coupling device on the head of the bone anchoring element. Thus, an in-situ placement of the coupling device onto a head of an inserted bone anchoring element can be achieved more gently without exerting excessive forces that may cause injuries.

In addition, the clamping force that the coupling device exerts on the head of the bone anchoring element may be increased. Also, inadvertent detachment of the locking ring from the receiving part may be avoided.

The coupling device permits, once placed onto the head of the bone anchor, a temporary locking without the requirement of a rod or an additional locking member. The rod can even be inserted and is not required to sit on the rod support but can be at an elevated position. Hence, by moving the locking ring from a locking position to a pre-locking position and vice versa, the bone anchoring device can be locked and unlocked to permit various adjustment steps. As such, the rod and/or the locking member are only truly needed for the final locking at the end of the surgical procedure. Therefore, the in-situ placement of the coupling device with the capability of temporary and/or variable locking of the head increases the variety of correction steps that can be carried out during surgery.

A polyaxial bone anchoring device according to an embodiment of the invention includes, in addition to the coupling device, a bone anchoring element having a head and a shank. The head may have a spherically-shaped outer surface portion.

The bone anchoring element may also be assembled with the coupling device prior to insertion into bone. Hence, the surgeon has a wide variety of options when handling the polyaxial bone anchoring device according to embodiments of the invention.

According to another embodiment of the invention, a coupling device for coupling a rod to a bone anchoring element that has a shank and a head includes a receiving part having a first end, a second end, a central axis extending between the first end and the second end, and a passage extending from the first end to the second end. The receiving part further includes a rod receiving portion having a recess defining a channel for receiving the rod, the recess extending from the first end towards the second end to a bottom and forming two free legs, and a head receiving portion open to the second end and including a seat portion for pivotably receiving the head of the bone anchoring element, the head receiving portion being flexible for inserting and clamping the head. The coupling device further includes a locking ring configured to be arranged around the head receiving portion. When the locking ring is around the head receiving portion, the locking ring can assume a locking position where the locking ring exerts a force on the head receiving portion to lock an inserted head. The head receiving portion includes, adjacent to the second end, an inner conical surface portion that narrows towards the seat portion and that has an axial height corresponding to an axial height of a portion of an inserted head that projects out of the seat portion, measured to the shank when the shank is coaxial with the central axis.

### BRIEF DESCRIPTION OF THE DRAWINGS

Further features and advantages of the invention will become apparent from the description of embodiments by means of the accompanying drawings. In the drawings:

FIG. 1 shows an exploded perspective view of a polyaxial bone anchoring device with a coupling device and a rod according to a first embodiment.

FIG. 2 shows a perspective view of the polyaxial bone anchoring device of FIG. 1 in an assembled state.

FIG. 3 shows a cross-sectional view of the polyaxial bone anchoring device of FIGS. 1 and 2, the cross-section taken in a plane perpendicular to a rod axis of an inserted rod.

FIG. 4 shows a perspective view from a top of a receiving part of the coupling device of FIGS. 1 to 3.

FIG. 5 shows a perspective view from a bottom of the receiving part of FIG. 4.

FIG. 6 shows a top view of the receiving part of FIGS. 4 and 5.

FIG. 7 shows a cross-sectional view of the receiving part of FIGS. 4 to 6, the cross-section taken along line A-A in FIG. 6.

FIG. 8 shows a perspective view from a top of a locking ring of the coupling device of FIGS. 1 to 3.

FIG. 9 shows a perspective view from a bottom of the locking ring of FIG. 8.

FIG. 10 shows a top view of the locking ring of FIGS. 8 and 9.

FIG. 11 shows a cross-sectional view of the locking ring of FIGS. 8 to 10, the cross-section taken along line B-B in FIG. 10.

FIGS. 12 to 15 show cross-sectional views of steps of assembling the polyaxial bone anchoring device of FIGS. 1 to 3 and locking of a head of a connected bone anchoring element, wherein the cross-sections are taken in a plane perpendicular to an axis of extension of a rod recess of the receiving part.

### DETAILED DESCRIPTION

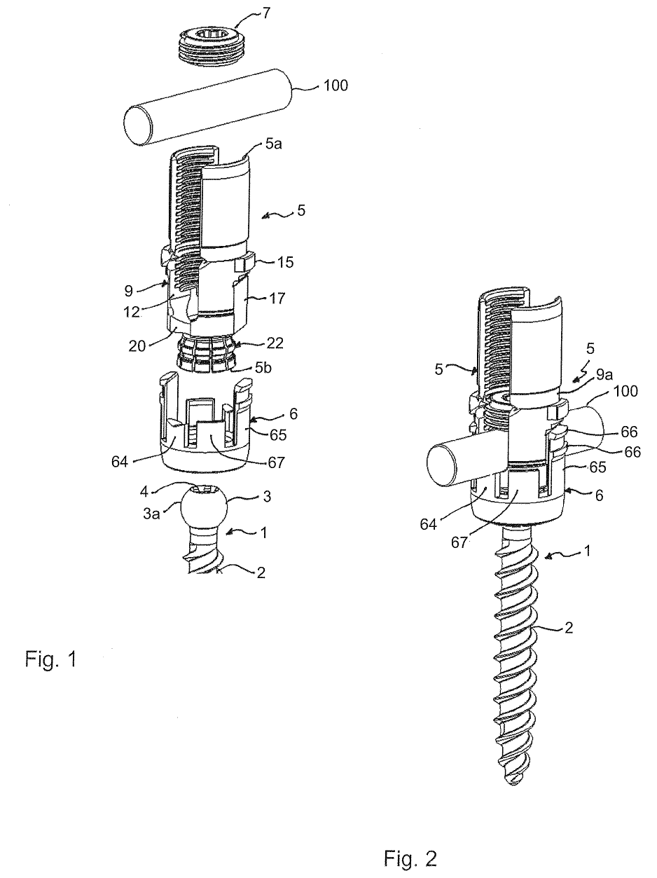

As shown in FIGS. 1 to 3, a polyaxial bone anchoring device according to an embodiment of the invention includes a bone anchoring element 1 in the form of, for example, a bone screw having a shank 2 with a threaded portion and a head 3 with a spherically-shaped outer surface portion 3a. The head 3 has a recess 4 for engagement with a driver or tool. The polyaxial bone anchoring device also includes a coupling device including a receiving part 5 for receiving a rod 100 to be connected to the bone anchoring element 1 and a locking ring 6. The locking ring 6 is configured to extend around a portion of the receiving part 5 for clamping and locking the head 3 in the receiving part 5. Further, a fixation element 7 in the form of, for example, an inner screw or set screw, may be provided for fixing the rod 100 in the receiving part 5. The polyaxial bone anchoring device is configured to permit coupling of the rod 100 to the bone anchoring element 1 at various angular positions of the coupling device relative to the shank 2 of the bone anchoring element 1.

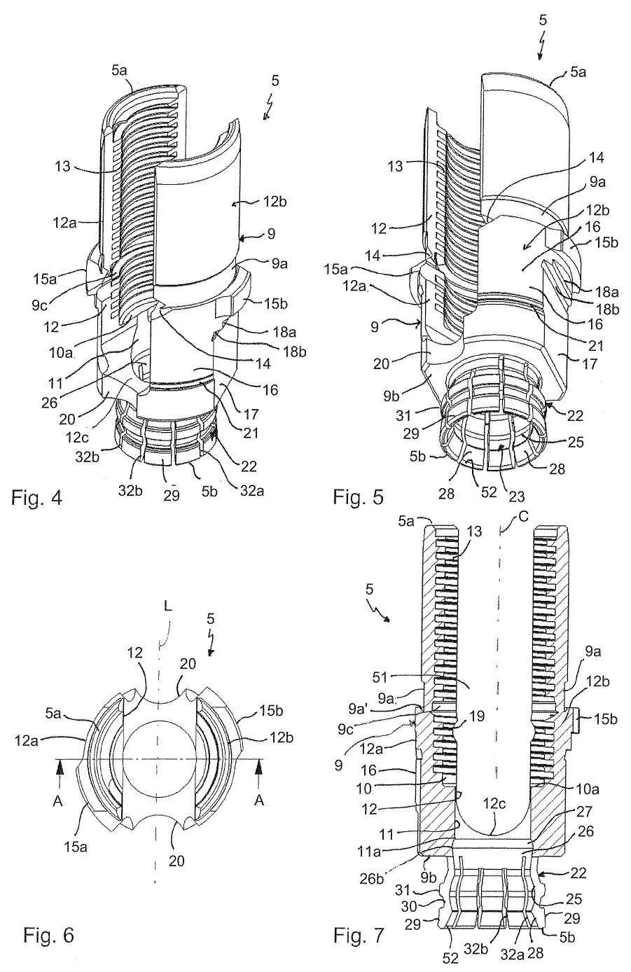

Referring additionally to FIGS. 4 to 7, the receiving part 5 may preferably be a monolithic part, and has a first end or top end 5a, a second end or bottom end 5b, and a passage 51 extending from the top end 5a towards the bottom end 5b, the passage 51 defining a longitudinal central axis C. By the passage 51, an opening 52 at the bottom end 5b is defined, which has a width that is greater than a greatest width of the head 3 of the bone anchoring element 1, so that the head 3 is insertable through the opening 52. The receiving part 5 includes a rod receiving portion 9 and a head receiving portion 22. Adjacent to the top end 5a, the receiving part 5 forms the rod receiving portion 9, which may have an outer shape that is substantially cylindrical. At a distance from the top end 5a, the rod receiving portion has a bottom end 9b. A coaxial first bore 10 is provided in the rod receiving portion 9 that extends from the top end 5a towards a first distance from the bottom end 9b of the rod receiving portion. The rod receiving portion 9 further includes a coaxial second bore 11 extending from a lower end of the first bore 10 to a second distance from the bottom end 9b of the rod receiving portion. A diameter of the second bore 11 may be smaller than a diameter of the first bore 10 and smaller than a diameter of the head 3 of the bone anchoring element 1.

A substantially U-shaped recess 12 extends from the top end 5a of the receiving part in the direction of the bottom end 9b in the rod receiving portion 9, wherein a width of the recess 12 is slightly larger than a diameter of the rod 100, such that the rod 100 can be placed in the recess 12 and can be guided therein. The recess 12 forms a channel for the rod 100. By means of the recess 12, two free legs 12a, 12b are formed, on which an internal thread 13 may be provided. The internal thread 13 can be, for example, a metric thread, a flat thread, a negative angle-thread, a saw-tooth thread, or any other thread form. Preferably, a thread form such as a flat thread or a negative angle thread is used to prevent or reduce splaying of the legs 12a, 12b when the fixation element 7 is screwed into the recess 12. It shall be noted that the internal thread 13 extends to a small distance from a bottom 10a of the bore 10, and that there is no undercut provided between the lower end of the internal thread 13 and the bottom 10a of the bore 10. Hence, the inside of the rod receiving portion 9 below the internal thread 13 is free from an undercut. This results in an enhanced wall strength in the lower region of the legs 12a, 12b, which renders them stiffer.

At a distance from the top end 5a, an external groove 9a or otherwise weakened section is provided that has a reduced wall thickness and that allows breaking off of the upper portion of the legs 12a, 12b from the rest of the receiving part. The external groove 9a may have a lower wall 9a′ which extends at an acute angle with respect to the central axis C, as best seen in FIG. 7. At an edge of the external groove 9a in a circumferential direction, recesses or cutouts 14 may be formed at either side of the rod channel and at both ends in the longitudinal direction of the rod channel. The cutouts 14 may facilitate breaking off of the upper portion of the legs 12a, 12b. Inside the bore 10, the internal thread 13 may be interrupted by means of an internal groove 9c at an axial position that substantially corresponds to a lower edge of the external groove 9a. Hence, the leg portions above the external groove 9a can serve as extended tabs. With the extended tabs, there is more axial space to manipulate the polyaxial bone anchoring device with respect to an inserted rod, which may be positioned, for example, at a higher position with respect to a bottom 12c of the recess 12. By means of this, for example, a vertebra can be pulled against the rod.

The depth of the recess 12 may be such that when the rod 6 is placed into the recess 12 and the fixation element 7 is screwed between the legs 12a, 12b, the locking element 7 does not substantially protrude out of the receiving part 5 when the extended tabs have been broken off.

At an outer surface of the rod receiving portion 9, an engagement structure for engagement with an instrument (not shown) may be provided. The engagement structure may include circumferentially extending ribs, which may be arranged asymmetrically with respect to a plane including the central axis C of the receiving part 5 and a channel axis L of the substantially U-shaped recess 12. In such an arrangement, a first rib 15a can start at a distance from the edge of the U-shaped recess 12 on one side of the receiving part and extend circumferentially for a distance around the rod receiving portion 9. A second rib 15b can start at a distance from the edge of the U-shaped recess on an opposite side of the receiving part and also extend circumferentially for a distance around the rod receiving portion 9. More specifically, the respective positions of the ribs 15a, 15b are offset from one another by 180° measured around the central axis C, and are rotated with respect to the central axis C, such that the rib 15a of the leg 12a is positioned adjacent to the rod receiving recess 12 at one side of the receiving part 5, and the rib 15b is positioned adjacent to the rod receiving recess 12 at the other side of the receiving part 5. Thereby, a rib-free surface 16 is formed on each side of the U-shaped recess 12. This permits an instrument to be placed first onto the rib-free surface, and then to be rotated to engage the ribs 15a, 15b. The ribs 15a, 15b may have a substantially rectangular cross-section and may have inclined end portions. It shall be understood that more than one rib can be provided in the axial direction. Also, the shape of the ribs may be different in other embodiments. Below each of the ribs 15a, 15b, a substantially flat outer surface portion 17 may be formed that may have a contour of a rectangle or square and that may serve for engagement with a portion of the locking ring 6. Between the ribs 15a, 15b and the substantially flat surface portion 17, two latching grooves 18a, 18b may be provided, one after the other in an axial direction. The latching grooves 18a, 18b are configured to cooperate with a portion of the locking ring 6 to provisionally hold the locking ring 6 at one or more specific positions. The upper latching groove 18a may serve for preventing further upward movement of the locking ring at an insertion position. The lower latching groove 18b may serve for latching the locking ring at a pre-locking position.

At an axial position slightly below the internal groove 9c and above the bottom 10a of the bore 10, an engagement structure 19, for example, in the form of a shallow groove, for engagement with an instrument, may be provided on the inner surface of the bore 10 and/or the recess 12.

Furthermore, cutouts 20 may be provided on either side of the substantially U-shaped recess 12, which may serve for receiving projections of the locking ring 6 therein. By means of this, the locking ring 6 can be secured against rotation relative to the receiving part 5. The cutouts 20 have a rounded, preferably a cylinder segment-shaped contour. Compared to a rectangular or square-shaped cutout, the rounded cutout 20 results in an enhanced stiffness of the receiving part 5, and in particular, of the rod receiving portion 9.

Moreover, a circumferentially extending groove 21 may be formed on the outer surface of the rod receiving portion 9 at an axial position above the cutouts 20 and below the ribs 15a, 15b. The groove 21 may serve as a further abutment for the locking ring 6.

The head receiving portion 22 extends from the bottom end 9b of the rod receiving portion to the bottom end 5b of the receiving part 5. The head receiving portion 22 has a substantially cap-like shape, with a hollow interior portion 23 providing a seat for receiving the head 3 pivotably therein. A greatest outer diameter of the head receiving portion 22 is smaller than a greatest outer diameter of the rod receiving portion 9. A free end of the head receiving portion 22 forms the bottom end 5b of the receiving part 5, with the passage 51 ending in the opening 52. In greater detail, the passage 51 forms a seat portion 25 for the head in the head receiving portion 22, which preferably has a spherical shape adapted to the shape of the spherically-shaped portion 3a of the head 3. Moreover, the seat portion 25 is configured to encompass the head 3 of the bone anchoring element 1 from the side, to cover a region of the head 3 including a largest diameter of the head 3.

Between the seat portion 25 and the second bore 11 of the rod receiving portion, a widening section 26 that widens towards the rod receiving portion 9, and preferably a conically widening section, is formed. The widening section 26 extends into the rod receiving portion 9. Adjacent to the seat portion 25, the widening section 26 has an inner diameter substantially matching an inner diameter of the seat portion 25 at this position. At its upper end, the widening section 26 has an inner diameter that is smaller than an inner diameter of the first bore 10 but greater than an inner diameter of the second bore 11. Between the lower end of the second bore 11 and the widening section 26, a small transition section 27 is formed that narrows towards the second bore 11. The transition section 27 ends a small distance from the bottom 12c of the U-shaped recess 12. The size of the widening section is designed such that the strength of the receiving part 5 is increased. In particular, the stiffness of the legs 12a, 12b may be increased.

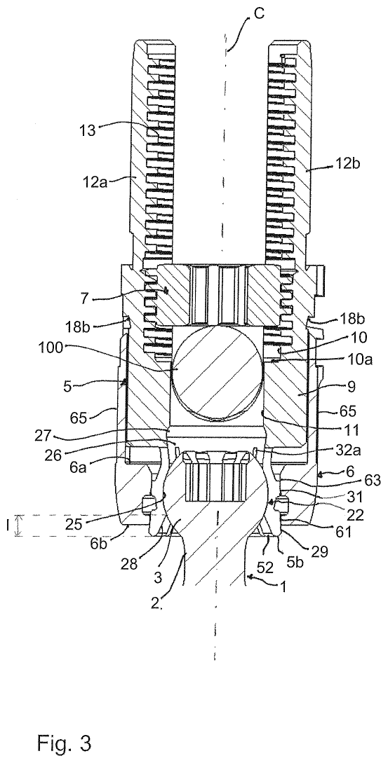

Adjacent to the bottom end 5b of the receiving part 5, the passage 51 has a widening section 28, preferably a conically widening section, that widens towards the bottom end 5b of the receiving part 5. The widening section 28 forms an angle with the central axis C. This angle defines the maximum pivot angle that the shank 2 of the bone anchoring element 1 can form with the central axis C. For example, the angle may be 30° or more. As best seen in FIG. 3, an axial length or height l of the widening section 28 may correspond substantially to an axial height of the portion of the head 3 that extends out of the seat portion 25 down to the shank 2, when the head 3 is received in the seat portion 25 and the shank 2 is coaxial with the central axis C. With such an axial length or height l of the widening section 28, the head receiving portion 22 projects below the locking ring 6 when the locking ring 6 is assembled to the receiving part 5, to an extent such that inadvertent moving of the locking ring 6 past the bottom end 5b of the receiving part 5 may be avoided.

An outer surface of the head receiving portion 22 may be recessed in a radial direction relative to the bottom end 9b of the rod receiving portion 9. The outer surface of the head receiving portion 22 has a circumferentially extending lowermost outer section 29 that is conically widening in a direction of the bottom end 5b of the receiving part 5. Preferably, the angle of the lowermost outer section 29 is about 4° to 6°, preferably about 5°, more preferably 5°, relative to the central axis C. The lowermost outer section 29 is configured to cooperate with a corresponding portion of the locking ring 6 to exert a compression force onto an inserted head. The axial length or height of the lowermost outer section 29 may correspond to the axial length or height of the widening section 28 of the hollow interior 23. Following the lowermost outer section 29, a circumferential groove 30 is formed which is followed by a circumferentially extending and radially outwardly bulged section 31. The radially outwardly bulged section 31 is configured to cooperate with another portion of the locking ring 6 to further exert a clamping force onto an inserted head 3. The bulged section 31 narrows as it extends in a direction towards the rod receiving portion 9, which contributes to the recessed shape of the head receiving portion 22.

A plurality of slits 32a, 32b render the head receiving portion 22 flexible, so that, when the head 3 is inserted into the hollow interior 23, the head receiving portion 22 expands. When pressure is exerted onto an inserted head 3 by the locking ring 6, the head receiving portion 22 is compressed. By means of this, the head 3 can be clamped or locked depending on the friction force between the head 3 and the seat portion 25. The slits 32a, 32b are open towards the bottom end 5b of the receiving part 5. A first type of slits 32a extend into the widening section 26 up to about an axial position of the bottom end 9b of the rod receiving portion 9. A second type of slits 32b, which are shorter than the first type of slits 32a, extend only slightly above the upper end of the seat portion 25. The second type of slits 32b may be arranged circumferentially at both sides of the center of the U-shaped recess 12. In the embodiment, four shorter slits 32b are provided, two on either side of the U-shaped recess 12. The first type of slits 32a may be arranged at the positions of the legs 12a, 12b, respectively, as can be seen in FIGS. 4, 5 and 7. The shorter slits may enhance the strength of the head receiving portion 22, and consequently the clamping force. The number of slits may vary in different embodiments according to the desired flexibility of the head receiving portion 22.

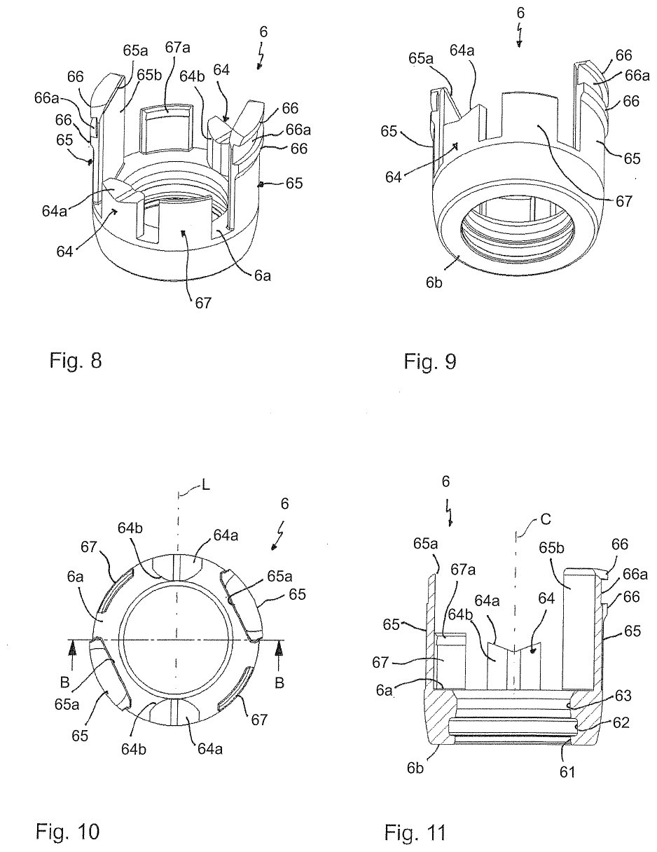

Turning now to FIGS. 8 to 11, the locking ring 6 will be described. The locking ring 6 is designed to encompass the head receiving portion 22, and has an internal surface structure that facilitates, in cooperation with the head receiving portion 22, a full locking of an inserted head 3 in the head receiving portion 22 when the locking ring 6 is at its lowermost position. The locking ring 6 further enables a pre-locking when the locking ring 6 is at a position slightly above the locking position, which still allows pivoting of the head 3 in the head receiving portion 22, but prevents removal of the head 3 from the head receiving portion 22. Lastly, the locking ring 6 is configured to permit insertion of the head 3 into the hollow interior 23 when the locking ring is at an insertion position, which is defined by an uppermost position the locking ring can assume relative to the receiving part 5.

In greater detail, the locking ring 6 has a lower surface 6b and an opposite upper surface 6a that is substantially ring-shaped. Adjacent to the lower surface 6b, there is a first annular projection 61, which projects radially inwardly and is configured to cooperate with the lowermost outer section 29 of the head receiving portion 22. The annular projection 61 may have a cylindrical inner surface or a slightly tapered inner surface. Following the annular projection 61, there is a widened section 62, which is then followed towards the upper surface 6a by a bulged section 63 that also projects radially inwardly. As depicted in FIG. 3, when the locking ring 6 is mounted around the head receiving portion 22, the first annular projection 61 is configured to press onto the lowermost outer section 29 of the head receiving portion 22, and the bulged section 63 is configured to press onto the bulged section 31 of the head receiving portion 22.

From the upper surface 6a of the ring-shaped portion of the locking ring 6, two rod support projections 64 protrude upwardly and have a free end surface forming a rod support surface 64a for the rod 100. An inner surface 64b of the projections 64 is rounded so as to mate with the shape of the cutouts 20, preferably, the inner surface 64b is cylindrical segment-shaped. The rod support projections 64 are diametrically opposite, i.e., offset by 180°, from one another. A cross-section of the rod support surface 64 a may be substantially V-shaped to permit safe support of rods of different diameters. When the locking ring 6 is mounted on the receiving part 5 such that the upper surface 6a faces towards the top end 5a of the receiving part, the rod support projections 64 may extend through the cutouts 20 in the rod receiving portion 9, and thereby secure the rotational orientation of the locking ring 6 relative to the receiving part 5.

The locking ring 6 also includes two upstanding arms 65 that are positioned asymmetrically with respect to a plane that extends through the central axis C and through the centers of the rod support surfaces 64a, for example, in the same or similar manner as the ribs 15a, 15b of the rod receiving portion 9 are arranged on the receiving part 5. At an upper end of the arms 65, an engagement portion in the form of circumferential ribs 66 defining a groove 66a are provided for engagement with an instrument. An inwardly facing upper edge 65a of the arms 65 may be provided that is configured to engage the latching grooves 18a, 18b at the receiving part 5. In the assembled state, the engagement structures in the form of the ribs 66 with the groove 66a are aligned with the engagement structures 15a, 15b at the receiving part 5, leaving the rib-free surface 16 of the receiving part 5 exposed. Moreover, the arms 65 have a substantially flat inner wall 65b that is configured to engage the flat portion 17 at the rod receiving portion 9. Thereby, a further form-fit connection is established between the locking ring 6 and the receiving part 5. Between the rod support projections 64 and the arms 65, there are upstanding wall portions 67 that have a height that is slightly greater than that of the rod support projections 64, and which have, at an inner wall thereof, a ledge 67a that may engage the groove 21 at the head receiving portion 9 of the receiving part 5.

A bone anchoring device according to an embodiment may further include the rod 100 that is configured to be inserted into the substantially U-shaped recess 12 and the fixation element 7, such as a set screw, that is configured to be screwed between the legs 12a, 12b. Thus, the fixation element 7 has a thread corresponding to the internal thread 13 provided on the legs 12a, 12b.

The coupling device and parts thereof, as well as the bone anchoring element and the locking element and the rod, may be made of any bio-compatible material, preferably, however, of titanium or stainless steel, or of any other bio-compatible metal or metal alloy or plastic material. For bio-compatible alloys, a NiTi alloy, for example Nitinol, may be used. Other materials that can also be used are, for example, magnesium or magnesium alloys. Bio-compatible plastic materials that can be used may be, for example, polyether ether ketone (PEEK) or poly-L-lactide acid (PLLA). The coupling device and the other parts of the polyaxial bone anchoring device may be made of the same or of different materials.

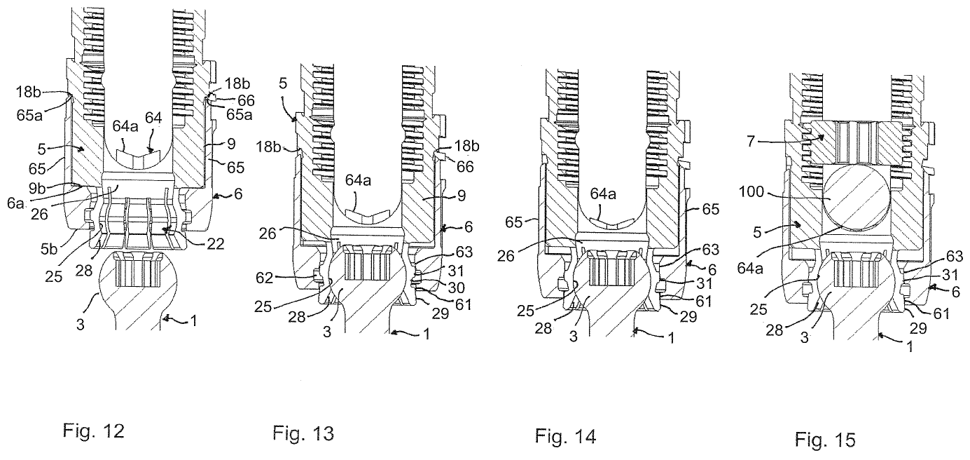

Steps of assembling the coupling device with the bone anchoring element 1 according to an embodiment of the invention will be described with reference to FIGS. 12 to 15.

The coupling device may include the receiving part 5 and the locking ring 6 in a pre-assembled manner. It shall be noted that the locking ring 6 can be mounted on the receiving part 5 from the bottom end 5b by compressing the head receiving portion 22. When mounted, the rod support projections 64 extend into the cutouts 20 of the receiving part 5. The bone anchoring element 1 may be placed already into bone, so that the coupling device can be assembled with the bone anchoring element in-situ. Alternatively, the coupling device can be assembled with the bone anchoring element 1 prior to insertion into bone.

As shown in FIG. 12, the locking ring 6 is in the insertion position in which the annular projection 61 of the locking ring 6 is positioned at approximately a same axial position as the groove 30 in the outer surface of the head receiving portion 22. The upper surface the arms 65 of the locking ring 6 may engage the upper groove 18a (generally located axially higher than the lower groove 18b but not circumferentially located and therefore not shown in the cross-sections in FIGS. 12 to 15) at the outer surface of the rod receiving portion 9. In the insertion position, the head 3 can be inserted into the hollow interior 23 of the head receiving portion 22. Due to the flexibility of the head receiving portion 22, the head receiving portion snaps over the head 3 such that the head 3 is seated in the seat portion 25 and may be held there by friction in some embodiments.

As shown in FIG. 13, the locking ring 6 is moved slightly downward towards the bottom end 5b of the receiving part 5 until the arms 65 snap into the lower groove 18b at the rod receiving portion 9. The locking ring 6 is now latched at a pre-locking position. In the pre-locking position, the annular projection 61 of the locking ring 6 presses on an upper portion of the lowermost outer section 29 of the head receiving portion 22, and the bulge section 63 of the locking ring 6 slightly presses onto the outwardly bulged section 31 of the head receiving portion 22. The head receiving portion 22 is compressed to an extent such that the head 3 cannot escape through the lower opening 52. The head may be held by friction in the seat portion 25, for example, caused by the compression by the locking ring 6, so that the coupling device can assume an angular position relative to the shank which can be maintained provisionally prior to locking.

Next, as depicted in FIG. 14, the locking ring 6 can be moved further downward, for example, by an instrument (not shown). Due to the increasing compression force acting onto the head receiving portion 22 when the annular projection 61 of the locking ring slides along the widening lowermost outer surface portion 29 of the head receiving portion 22, the head 3 is clamped in the head receiving portion 22. The clamping force can have a strength such that the head may be locked temporarily therein.

In the configuration shown in FIG. 14, when moving the locking ring 6, for example, with an instrument, upwards out of the temporary locking position, for example, back towards the pre-locking position shown in FIG. 13, the head can be unlocked from temporary locking and may be pivoted again. Locking and unlocking can be carried out with the instrument several times. Neither a rod nor a fixation member needs to be present in the rod channel for locking and unlocking of the head relative to the coupling device.

Finally, when a suitable angular position of the polyaxial bone anchoring device is found, the rod 100 may be inserted and the fixation member 7 may be placed between the legs 12a, 12b and tightened to finally lock the head, as shown in FIG. 15. Finally, the upper portions of the legs 12a, 12b which served as extended tabs are broken-off.

In clinical use, a plurality of polyaxial bone anchoring devices are inserted into bone parts or into vertebrae, in particular, into the pedicles of vertebrae. The coupling devices are then aligned so that a rod (not shown) can be received in the rod receiving portions of two or more of the bone anchoring devices.

Modifications of the above described embodiments are also conceivable. In particular, the shapes of the parts is not limited to the detailed shapes shown in the figures. Deviations may be possible and encompassed by the disclosure.

Instead of the locking member being a set screw, all other kinds of locking assemblies known in the art may be used. For the bone anchoring element, all types of bone anchoring elements that are suitable for anchoring in bone or a vertebra, such as bone screws, bone nails, etc., may be used. The rod can be any elongate device that is configured to connect two bone anchoring devices. The rod may have various shapes and/or varying cross-sections along its length. The rod may be stiff or more flexible.

The spherical outer surface portion of the head may be only partly spherical, for example, when seen in the circumferential direction, and the seat portion in the head receiving portion may be adapted thereto, such that pivoting of the head may only be possible, for example, in a predefined plane.

Moreover, the head receiving portion may have a design that allows pivoting of the bone anchoring element to a greater pivot angle to one side or radial direction relative to the coupling device compared to other sides or radial directions.

While the present invention has been described in connection with certain exemplary embodiments, it is to be understood that the invention is not limited to the disclosed embodiments, but is instead intended to cover various modifications and equivalent arrangements included within the spirit and scope of the appended claims, and equivalents thereof.

What is claimed is:

1. A coupling device for coupling a rod to a bone anchoring element that comprises a head and a shank for anchoring to bone, the coupling device comprising:
a receiving part having a first end, a second end below the first end, a central axis extending between the first and second ends, a rod receiving portion having a recess that extends from the first end to a bottom towards the second end for receiving the rod and that forms two free legs, and a flexible head receiving portion open to the second end to facilitate insertion and clamping of the head of the bone anchoring element therein, the head receiving portion comprising a seat for pivotably holding the head; and
a locking ring positionable around the head receiving portion and configured to assume a locking position where the locking ring exerts a force on the head receiving portion to lock the head therein;
wherein a passage extends through the receiving part from the first end to the seat, the passage comprising a widening section that widens as the widening section extends axially upwardly from a lower end closer to the seat to an upper end closer to but positioned axially lower than the bottom of the recess, and wherein an inner diameter of the upper end of the widening section is greater than an inner diameter of another section of the passage that is located axially higher than the bottom of the recess.

2. The coupling device of claim 1, wherein the widening section has a conical segment-shaped profile.

3. The coupling device of claim 1, wherein between the upper end of the widening section and the bottom of the recess, the passage further comprises a transition section that narrows as the transition section extends axially upwardly towards the bottom of the recess.

4. The coupling device of claim 3, wherein the transition section narrows continuously as the transition section extends axially upwardly towards the bottom of the recess.

5. The coupling device of claim 1, wherein the head receiving portion comprises an outer conical surface at the second end of the receiving part that widens towards the second end at an angle between about 4° and about 6° relative to the central axis.

6. The coupling device of claim 1, wherein the head receiving portion defines a plurality of axially extending slits that are open towards the second end of the receiving part, and wherein at least one of the slits extends to an axial position that is closer to the upper end of the widening section than to the lower end of the widening section.

7. The coupling device of claim 6, wherein the at least one of the slits is positioned circumferentially away from the recess of the rod receiving portion.

8. The coupling device of claim 1, wherein the head receiving portion defines a plurality of axially extending slits that are open towards the second end of the receiving part, and wherein at least one of the slits extends to an axial position that is closer to the lower end of the widening section than to the upper end of the widening section.

9. The coupling device of claim 8, wherein the at least one of the slits is circumferentially aligned with the recess of the rod receiving portion.

10. The coupling device of claim 1, wherein each of the legs comprises an extended tab that extends axially away from the second end of the receiving part and that is separable from the rest of the leg.

11. The coupling device of claim 1, wherein the passage forms an inner thread adjacent to the first end to engage a fixation element for locking a rod in the recess of the receiving part.

12. The coupling device of claim 11, wherein the rod receiving portion is free from any undercut positioned below the inner thread.

13. A bone anchoring device comprising the coupling device of claim 1 and the bone anchoring element comprising the shank and the head.

14. The bone anchoring device of claim 13, wherein the head comprises a spherical segment-shaped outer surface to facilitate a polyaxial connection with the coupling device.

15. The bone anchoring device of claim 13, wherein when the head is held in the seat and the shank is coaxial with the central axis of the receiving part, the head extends axially downwardly past the seat by a first axial distance, and wherein the head receiving portion comprises an inner conical surface at the second end of the receiving part that widens as the inner conical surface extends downwardly away from the seat, the inner conical surface having an axial height that is at least the first axial distance.

16. The bone anchoring device of claim 15, wherein the inner conical surface forms an angle of at least 30° relative to the central axis.

17. The bone anchoring device of claim 13, wherein the rod receiving portion has an outer surface and a first engagement surface formed on the outer surface of the rod receiving portion, and the locking ring has an outer surface and a second engagement surface formed on the outer surface of the locking ring, and wherein when the head is held in the seat, the first and second engagement surfaces are configured to be simultaneously engaged by an instrument to move the locking ring away from the locking position to a position where the head is pivotable relative to the receiving part.

18. The bone anchoring device of claim 17, wherein the first and second engagement surfaces are further configured to be simultaneously engaged by the instrument to move the locking ring from the position where the head is pivotable to the locking position.

19. A method for coupling a rod to a bone using a bone anchoring device comprising a bone anchoring element comprising a head and a shank, a receiving part having a first end, a second end below the first end, a central axis extending between the first and second ends, a rod receiving portion having a recess that extends from the first end to a bottom towards the second end and that forms two free legs, and a flexible head receiving portion open to the second end to facilitate insertion and clamping of the head of the bone anchoring element therein, the head receiving portion comprising a seat for pivotably holding the head, a locking ring positionable around the head receiving portion, wherein a passage extends through the receiving part from the first end to the seat, the passage comprising a widening section that widens as the widening section extends axially upwardly from a lower end closer to the seat to an upper end closer to but positioned axially lower than the bottom of the recess, and wherein an inner diameter of the upper end of the widening section is greater than an inner diameter of another section of the passage that is located axially higher than the bottom of the recess, and a fixation element, the method comprising:
pivoting the receiving part relative to the head of the bone anchoring element to a desired position when the shank is anchored to bone and the head is held in the seat;
inserting the rod into the recess of the rod receiving portion; and
advancing the fixation element into the rod receiving portion to lock the rod and to urge the rod against the locking ring to move the locking ring to a locking position where the locking ring exerts a force on the head receiving portion to lock the head therein.

20. A coupling device for coupling a rod to a bone anchoring element that comprises a head and a shank for anchoring to bone, the coupling device comprising:
a receiving part having a first end, a second end below the first end, a central axis extending between the first and second ends, a rod receiving portion having a recess that extends from the first end to a bottom towards the second end for receiving the rod and that forms two free legs, and a flexible head receiving portion open to the second end to facilitate insertion and clamping of the head of the bone anchoring element therein, the head receiving portion comprising a seat for pivotably holding the head; and
a locking ring positionable around the head receiving portion and configured to assume a locking position where the locking ring exerts a force on the head receiving portion to lock the head therein, the locking ring comprising a projection configured to extend upwardly into the recess of the rod receiving portion to engage the rod;
wherein a cutout extends axially downwardly from bottom of the recess of the rod receiving portion to accommodate the projection of the locking ring, wherein for a cross-section taken in a plane perpendicular to the central axis, the cutout extends radially inwardly from an outer surface of the rod receiving portion to a bottom, where the bottom of the cutout has a concavely curved profile that curves towards the central axis.
